(19) United States
(12) Patent Application Publication
(10) Pub. No.: US 2024/0299600 A1
(43) Pub. Date: Sep. 12, 2024

(54) METHOD FOR TREATING ACUTE MYELOID LEUKEMIA USING VENETOCLAX IN CONJUNCTION WITH LINTUZUMAB-AC225

(71) Applicant: ACTINIUM PHARMACEUTICALS, INC., New York, NY (US)

(72) Inventor: Mark Berger, New York, NY (US)

(73) Assignee: ACTINIUM PHARMACEUTICALS, INC., New York, NY (US)

(21) Appl. No.: 18/044,356

(22) PCT Filed: Sep. 7, 2021
(86) PCT No.: PCT/US21/49225

§ 371 Date: Mar. 7, 2023

Related U.S. Application Data
(60) Provisional application No. 63/075,374, filed on Sep. 8, 2020.

(51) Int. Cl. A61K 51/10; A61K 9/00; A61K 31/635; A61P 35/02

(52) U.S. Cl. CPC A61K 51/1027; A61K 9/0019; A61K 9/0053; A61K 31/635; A61P 35/02

(57) ABSTRACT

Provided are methods for treating acute myeloid leukemia that include administering a regimen of venetoclax and lintuzumab-Ac225, in which (a) the regimen includes a plurality of cycles, each cycle lasting from 28 to 60 days, (b) the regimen includes (i) orally administering venetoclax on days 1 and 2 of the first cycle at a ramp-up dosage, and thereafter orally administering 400 mg of venetoclax daily on days 3-21 of the first cycle and days 1-21 of each subsequent cycle, and (ii) intravenously administering lintuzumab-Ac225 on day 4, 5, 6 or 7 of each cycle at a dose of from 0.1 μCi/kg to 2.0 μCi/kg, and (c) the subject has a peripheral blast burden at or below 1,000 blast cells/μl. Also provided are related methods in which the regimen includes intravenously administering lintuzumab-Ac225 twice during each cycle.

### CROSS-REFERENCE TO RELATED APPLICATIONS

This application is a 371 national stage of International Application No. PCT/US2021/049225 filed Sep. 7, 2021 which claims priority to U.S. Provisional Application Ser. No. 63/075,374 filed Sep. 8, 2020 which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Aug. 17, 2023 is named ATNM-005PCT_SL.txt and is 13,816 bytes in size.

### FIELD OF THE INVENTION

The present invention relates to the field of radiopharmaceuticals.

### BACKGROUND

### Acute Myeloid Leukemia

Acute myeloid leukemia (AML) is a heterogeneous group of neoplasms characterized by genetic abnormalities that impair differentiation and increase proliferation of hematopoietic cells. The symptoms of AML are caused by replacement of normal bone marrow with leukemia cells, which causes a decrease in red blood cells, platelets, and normal white blood cells. These symptoms include fatigue, shortness of breath, easy bruising and bleeding, and increased risk of infection. Several risk factors and chromosomal abnormalities have been identified, but the specific cause is not clear. As an acute leukemia, AML progresses rapidly and is typically fatal within weeks or months if left untreated.

The median age at diagnosis is 68 years. Among all cases of newly diagnosed AML (N=13,410), 55% (n=7,400) occur in individuals age 65 years and older. Despite modest improvements in the outcome of younger patients in recent decades, overall treatment results remain disappointing, especially in older patients. Data from the Surveillance, Epidemiology, and End Results (SEER) program of the National Cancer Institute show that the five-year survival rate for patients under age 65 years is approximately 45%. In contrast, five-year survival for patients 65 years and older is 6%.

Complete remission (CR), defined as less than 5% myeloblasts in the bone marrow, determined by cytomorphology and recovery of normal blood counts, has traditionally been the endpoint for evaluating treatment efficacy in AML. Patients who achieve this endpoint of CR were observed to have longer survival than other responses and the increase in survival corresponds directly with the duration of CR. However, leukemia cells that survive seemingly successful therapy may hide below the limit of cytomorphologic detection and cause relapse.

### Venetoclax

BCL-2 inhibitors have potential for treating malignancies. One such BCL-2 inhibitor is venetoclax, a drug that has been approved for treating chronic lymphocytic leukemia (“CLL”) (FDA News Release). Venetoclax binds to the BH3-binding groove of BCL-2, displacing pro-apoptotic proteins like BIM to initiate mitochondrial outer membrane permeabilization (“MOMP”), the release of cytochrome c, and caspase activation, ultimately resulting in programmed cancer cell death (i.e., apoptosis) (Venclexta™ Product Monograph).

Apoptosis is a mechanism of cell death in cancer cells, in addition to necrosis and autophagy (G. Kroemer, et al.). Ideally, by changing the balance between pro-apoptotic and anti-apoptotic stimuli, venetoclax would facilitate programed cell death of cancer cells and thus improve cancer patient outcomes.

However, apoptosis is a complex pathway. Cancer cells can develop various mechanisms to circumvent and/or abrogate a given treatment strategy intended to cause apoptotic death (S. Fulda) (as presented in FIG. 1 of that reference). For example, X-linked XIAP can abrogate the blocking of BCL-2. XIAP is a well-characterized inhibitor of apoptosis proteins (IAPs) (E. Shiozaki, et al.). Indeed, the majority of human cancers harbor high levels of IAPs such as XIAP (I. Tamm, et al.).

Other possible mechanisms of circumventing the effect of BCL-2 inhibitors include, for example, blocking activation of caspase 8 to prevent the downstream activity of venetoclax on the BAX/BCL-2 axis. Also, stimulating or un-blocking one part of the apoptotic pathway may not be sufficient to cause apoptosis, as pro-apoptotic stimuli are still needed to trigger an apoptotic pathway (D. Potter and A. Letai), (Venetoclax Advisory Committee Briefing Document).

Consequently, not all cancer cells respond to BCL-2 inhibitors. In one venetoclax trial, for example, the complete response rate (including complete responses with incomplete marrow recovery) was 7.5%, even though a majority of patients (79.4%) had some level of response to venetoclax (Venclexta™ Product Monograph). In addition, venetoclax has a significant myelosuppressive effect on neutrophils, with 40% of patients experiencing grade 3 and/or 4 neutropenia (Venclexta™ Product Monograph).

### Lintuzumab-Ac225

Radiation is a recognized way to treat cancer. It is known that cellular effects of radiation include cell cycle arrest, mutation, apoptosis, necrosis, and autophagy (J. Kiang, et al.). Radiation-related mediators of cellular damage include: (i) direct LED (linear energy deposition); (ii) ROS (reactive oxygen species); and (iii) RNS (reactive nitrogen species) (J. Kiang, et al.).

These mediators lead to cell damage/kill/arrest via the following mechanisms: (i) DNA damage (J. Kiang, et al.) (e.g., double-strand DNA breaks (most efficient), single-strand DNA breaks (less efficient, repairable), DNA base damage (least efficient, repairable), and DNA crosslinks); (ii) direct effects on the apoptotic cascade (e.g., direct activation of caspases, and damage to IAPs) (C. Friesen, et al.); and (iii) bystander effects (i.e., damage or killing of cells not directly damaged by radiation, which damage or killing occurs through mediation via gap junction communication and/or cytokines from target cells) (S. Sofou).

One important radioactive agent is lintuzumab-Ac225. It is an alpha-emitting isotope-labeled agent that targets cancer cells in the subject. Specifically, lintuzumab-Ac225 binds to CD33, a hematologic malignancy-associated antigen.

### SUMMARY OF THE INVENTION

The invention provides a method for treating a human subject afflicted with acute myeloid leukemia including administering to the subject a regimen of venetoclax and lintuzumab-Ac225, wherein (a) the regimen includes a plurality of cycles, each cycle lasting from 28 to 60 days, (b) the regimen includes (i) orally administering venetoclax on days 1 and 2 of the first cycle at a ramp-up dosage, and thereafter orally administering 400 mg of venetoclax daily on days 3-21 of the first cycle and days 1-21 of each subsequent cycle, and (ii) intravenously administering lintuzumab-Ac225 on day 4, 5, or 6 of each cycle at a dose of from 0.1 μCi/kg to 2.0 μCi/kg; and (c) the subject has a peripheral blast burden at or below 1,000 blast cells/μl.

The invention also provides a method for treating a human subject afflicted with acute myeloid leukemia including administering to the subject a regimen of venetoclax and lintuzumab-Ac225, wherein (a) the regimen includes four cycles, each cycle lasting 28 days, (b) the regimen includes (i) orally administering venetoclax at a dose of 100 mg and 200 mg, respectively, on days 1 and 2 of the first cycle, and thereafter orally administering 400 mg of venetoclax daily on days 3-21 of the first cycle and days 1-21 of each subsequent cycle, and (ii) intravenously administering lintuzumab-Ac225 on day 5 of each cycle at a dose of 0.25 μCi/kg, 0.5 μCi/kg, 1.0 μCi/kg, or 1.5 μCi/kg; and (c) the subject has a peripheral blast burden at or below 1,000 blast cells/μl.

The invention further provides a method for treating a human subject afflicted with acute myeloid leukemia including administering to the subject a regimen of venetoclax and lintuzumab-Ac225, wherein (a) the regimen includes a plurality of cycles, each cycle lasting from 28 to 60 days, (b) the regimen includes (i) orally administering venetoclax on days 1 and 2 of the first cycle at a ramp-up dosage, and thereafter orally administering 400 mg of venetoclax daily on days 3-21 of the first cycle and days 1-21 of each subsequent cycle, and (ii) intravenously administering lintuzumab-Ac225 on day 4, 5, or 6 of each cycle at a dose of from 0.05 μCi/kg to 1.0 μCi/kg, and on day 18, 19, or 20 of each cycle at a dose of from 0.05 μCi/kg to 1.0 μCi/kg; and (c) the subject has a peripheral blast burden at or below 1,000 blast cells/μl.

The invention still further provides a method for treating a human subject afflicted with acute myeloid leukemia including administering to the subject a regimen of venetoclax and lintuzumab-Ac225, wherein (a) the regimen includes four cycles, each cycle lasting 28 days, (b) the regimen includes (i) orally administering venetoclax at a dose of 100 mg and 200 mg, respectively, on days 1 and 2 of the first cycle, and thereafter orally administering 400 mg of venetoclax daily on days 3-21 of the first cycle and days 1-21 of each subsequent cycle, and (ii) intravenously administering lintuzumab-Ac225 on day 5 of each cycle at a dose of 0.125 μCi/kg, 0.25 μCi/kg, 0.5 μCi/kg, or 0.75 μCi/kg, and on day 19 of each cycle at a dose of 0.125 μCi/kg, 0.25 μCi/kg, 0.5 μCi/kg, or 0.75 μCi/kg; and (c) the subject has a peripheral blast burden at or below 1,000 blast cells/μl.

Additional features, advantages, and embodiments of the invention may be set forth or apparent from consideration of the following detailed description, drawings if any, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1 shows a schematic diagram of the expression plasmids for HuM195. The humanized VL and VH exons of HuM195 are flanked by XbaI sites. The VL exon was inserted into mammalian expression vector pVk, and the VH exon into pVg1 (Co, et al., J. Immunol. 148: 1149-1154, 1992).

FIG. 2 shows the complete sequence of the HuM195 light chain gene cloned in pVk between the XbaI and BamHI sites. The nucleotide number indicates its position in the plasmid pVk-HuM195. The VL and CK exons are translated in single letter code; the dot indicates the translation termination codon. The mature light chain begins at the double-underlined aspartic acid (D). The intron sequence is in italics. The polyA signal is underlined. The full-length nucleotide sequence and the encoded amino acid sequence are disclosed as SEQ ID NO: 1 and SEQ ID NO:2 respectively.

FIG. 3 shows the complete sequence of the HuM195 heavy chain gene cloned in pVg1 between the XbaI and BamHI sites. The nucleotide number indicates its position in the plasmid pVg1-HuM195. The VH, CH1, H, CH2 and CH3 exons are translated in single letter code; the dot indicates the translation termination codon. The mature heavy chain begins at the double-underlined glutamine (Q). The intron sequences are in italics. The poly A signal is underlined. The full-length nucleotide sequence and the encoded amino acid sequence are disclosed as SEQ ID NO:3 and SEQ ID NO:4 respectively.

FIG. 4 shows the structure of ²²⁵Ac-Lintuzumab (²²⁵Ac-HuM195).

FIG. 5 shows a flowchart for the production of ²²⁵Ac-Lintuzumab (²²⁵Ac-HuM195).

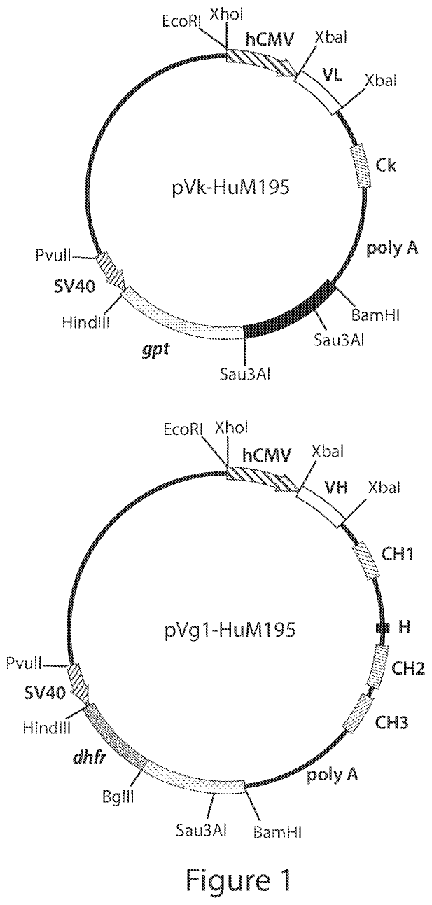

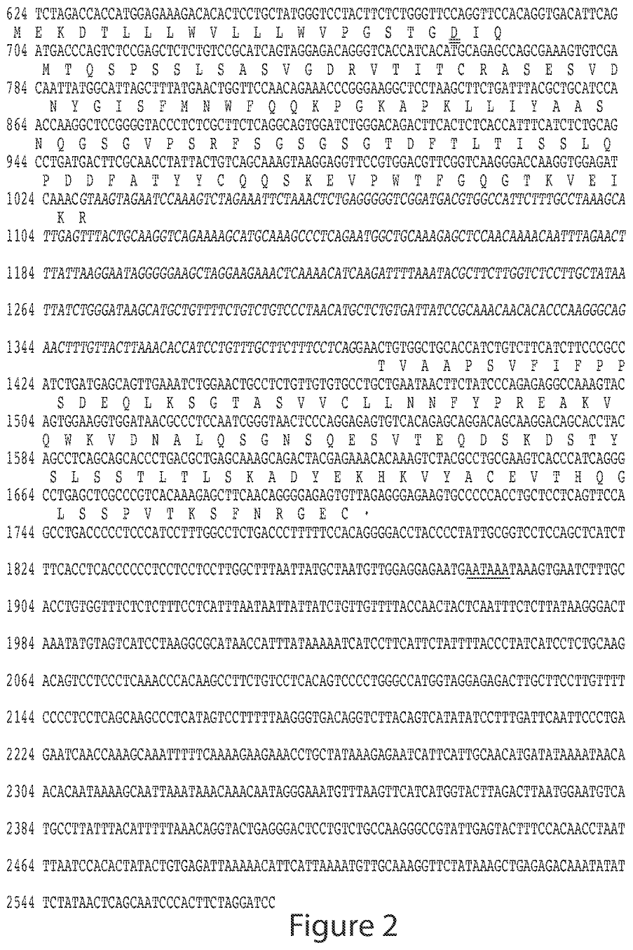

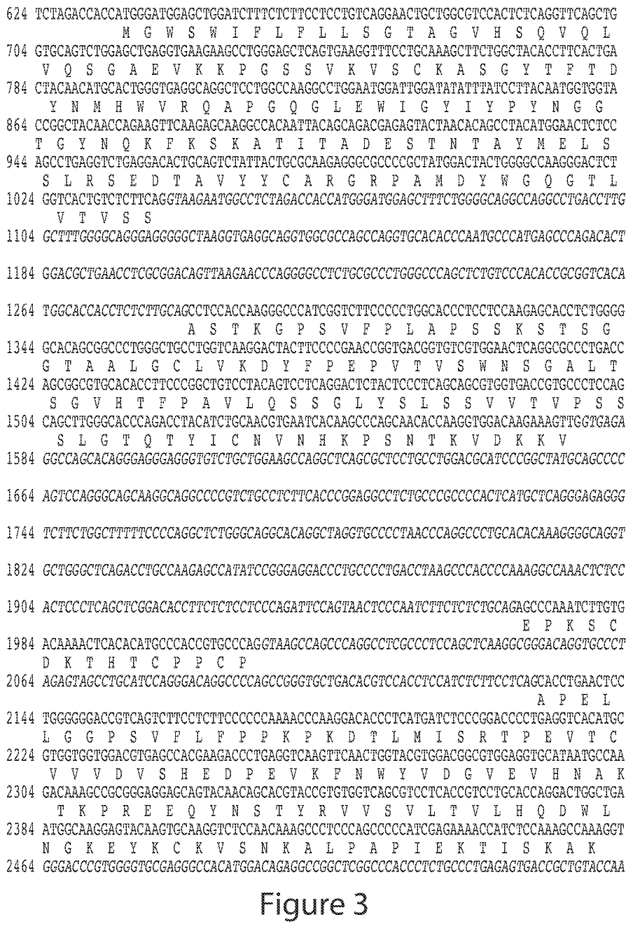

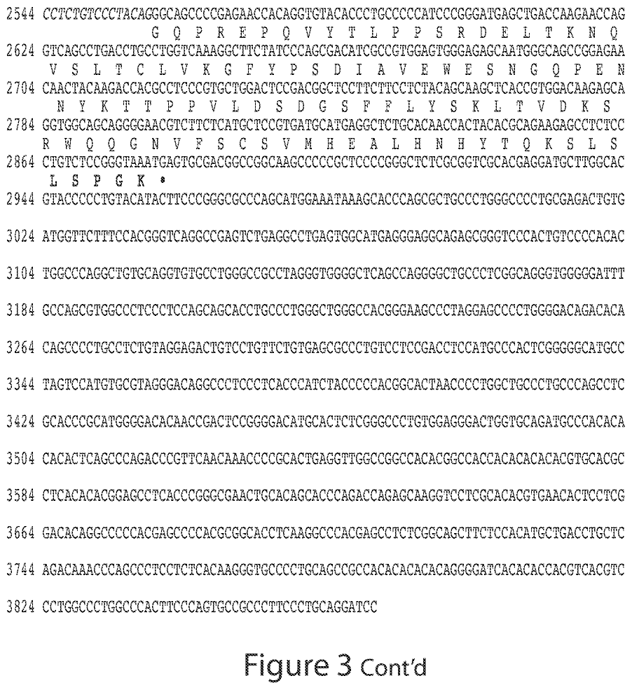

### DETAILED DESCRIPTION OF THE INVENTION

The invention provides methods, uses and pharmaceutical compositions for treating a human patient afflicted with acute myeloid leukemia (AML). These methods include administering to the subject certain regimens of venetoclax and lintuzumab-Ac225 in conjunction with one another.

### Definitions

In this application, certain terms are used which shall have the meanings set forth as follows.

As used herein, “acute myeloid leukemia” (also known as “acute myelogenous leukemia” and “AML”) includes, without limitation, the following: CD33-positive acute myeloid leukemia; CD33-negative acute myeloid leukemia; relapsed acute myeloid leukemia; non-relapsed acute myeloid leukemia; refractory acute myeloid leukemia; non-refractory acute myeloid leukemia; relapsed/refractory acute myeloid leukemia; and non-relapsed/refractory acute myeloid leukemia. In a preferred embodiment of the present invention, a subject afflicted with acute myeloid leukemia is afflicted with CD33-positive acute myeloid leukemia. In another preferred embodiment, a subject afflicted with acute myeloid leukemia is afflicted with relapsed/refractory acute myeloid leukemia. In a further preferred embodiment, a subject afflicted with acute myeloid leukemia is afflicted with CD33-positive relapsed/refractory acute myeloid leukemia.

As used herein, “administer”, with respect to an agent, means to deliver the agent to a subject's body via any known method. Specific modes of administration include, without limitation, intravenous, oral, sublingual, transdermal, subcutaneous, intraperitoneal, intrathecal and intra-tumoral administration.

In addition, in this invention, the antibodies used can be formulated using one or more routinely used pharmaceutically acceptable carriers. Such carriers are well known to those skilled in the art. For example, injectable drug delivery systems include solutions, suspensions, gels, microspheres and polymeric injectables, and can include excipients such as solubility-altering agents (e.g., ethanol, propylene glycol and sucrose) and polymers (e.g., polycaprylactones and PLGA's).

As used herein, the term “burden”, when used in connection with a cancerous cell, means quantity. So, a cancerous cell “burden” means the quantity of cancerous cells. Cancerous cells have a burden with respect to their tissue of origin (i.e., the primary site of disease), such as the “bone marrow blast burden” in the case of AML. Cancerous cells also have a burden with respect to one or more tissues other than those of origin, such as the blast burden in blood, liver, and spleen in the case of AML. The term “peripheral burden” relates to such cells. The peripheral burden of cancerous cells, such as blasts in the case of AML, can be measured in different ways with different outcomes. For example, in the case of AML, the “peripheral blast burden” can be measured as the total blast population outside of the bone marrow, or the total blast population of the blood, spleen, and liver combined, or simply the blast population of the blood as measured in cells per unit volume. As used herein in connection with AML, and unless stated otherwise, the term “peripheral blast burden” (i.e., peripheral cancerous cell burden) refers to the cancerous cell population of the blood as measured in cells per unit volume (e.g., cells/μl). This blood-based measurement is a useful proxy for the more cumbersome measurements of spleen and liver burdens, for example.

As used herein, the “human subject” can be of any age. For example, the subject can be 60 years old or older, 65 years old or older, 70 years old or older, 75 years old or older, 80 years old or older, 85 years old or older, or 90 years old or older. Alternatively, the subject can be 50 years old or younger, 45 years old or younger, 40 years old or younger, 35 years old or younger, 30 years old or younger, 25 years old or younger, or 20 years old or younger. For a human subject afflicted with AML, the subject can be newly diagnosed, or relapsed and/or refractory, or in remission.

“Lintuzumab” (which is the generic name for the antibody “HuM195”) is known, as are methods of making it. Likewise, methods of labeling lintuzumab with the radioisotope ²²⁵Actinium (²²⁵Ac) to make lintuzumab-Ac225 (also known as and referred to herein as ²²⁵Ac-lintuzumab, and ²²⁵Ac-HuM195) are known. These methods are exemplified, for example, in Scheinberg, et al., U.S. Pat. No. 6,683,162. This information is also exemplified in the examples and figures below. As used herein, “²²⁵Ac”, “225Ac”, and “Ac225” are synonymous.

As used herein, a “ramp-up dosage” of venetoclax is a dosage below 400 mg that is administered prior to administering a 400 mg dose of venetoclax. Ramp-up dosing lowers the risk of tumor lysis syndrome.

As used herein, “treating” a subject afflicted with a disorder shall include, without limitation, (i) slowing, stopping or reversing the disorder's progression, (ii) slowing, stopping or reversing the progression of the disorder's symptoms, (iii) reducing the likelihood of the disorder's recurrence, and/or (iv) reducing the likelihood that the disorder's symptoms will recur. In the preferred embodiment, treating a subject afflicted with a disorder means (i) reversing the disorder's progression, ideally to the point of eliminating the disorder, and/or (ii) reversing the progression of the disorder's symptoms, ideally to the point of eliminating the symptoms, and/or (iii) reducing or eliminating the likelihood of relapse (i.e., consolidation, which is a common goal of post-remission therapy for AML and, ideally, results in the destruction of any remaining leukemia cells).

The treatment of AML can be measured according to a number of clinical endpoints. These include, without limitation, survival time (such as weeks, months, or years of improved survival time, e.g., one, two or more months of additional survival time), and response status (such as complete remission (CR), complete remission with incomplete platelet recovery (CRp), complete remission with incomplete peripheral blood recovery (CRi), morphologic leukemia-free state (MLFS), and partial remission (PR)).

In one embodiment, the treatment of AML is measured in terms of remission. Included here are the following non-limiting examples. (a) Morphologic complete remission (“CR”): ANC≥1,000/μl, platelet count≥100,000/μl, <5% bone marrow blasts, no Auer rods, no evidence of extramedullary disease. (No requirements for marrow cellularity, hemoglobin concentration). (b) Morphologic complete remission with incomplete blood count recovery (“CRi”): Same as CR but ANC may be <1,000/μl and/or platelet count<100,000/μl. (c) Partial remission (PR): ANC≥1,000/μl, platelet count>100,000/μl, and at least a 50% decrease in the percentage of marrow aspirate blasts to 5-25%, or marrow blasts<5% with persistent Auer rods. These criteria and others are known, and are described, for example, in SWOG Oncology Research Professional (ORP) Manual Volume I, Chapter 11A, Leukemia (2014). An additional example is complete remission with partial hematologic recovery (CRh), defined as <5% bone marrow blasts, no evidence of disease, platelet count>50,000/μl, and ANC>500/μl.

### Embodiments of the Invention

In one embodiment, the invention provides a method for treating a human subject afflicted with acute myeloid leukemia including administering to the subject a regimen of venetoclax and lintuzumab-Ac225, wherein (a) the regimen includes a plurality of cycles, each cycle lasting from 28 to 60 days, (b) the regimen includes (i) orally administering venetoclax on days 1 and 2 of the first cycle at a ramp-up dosage, and thereafter orally administering 400 mg of venetoclax daily on days 3-21 of the first cycle and days 1-21 of each subsequent cycle, and (ii) intravenously administering lintuzumab-Ac225 on day 4, 5, or 6 of each cycle at a dose of from 0.1 μCi/kg to 2.0 μCi/kg; and (c) the subject has a peripheral blast burden at or below 1,000 blast cells/μl.

The regimen may, for example, include two cycles, three cycles, four cycles, five cycles, six cycles, seven cycles, eight cycles, nine cycles, or ten cycles. In one variation, the regimen includes four cycles. In another variation, the regimen includes eight cycles.

Each cycle may, for example, last 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days. Each cycle may, for example, last 28-30 days, such as 28 days. In another embodiment, the cycles may be of different durations. For example, each of the first two cycles of a four-cycle regimen may last 28 days, and each of the last two cycles of the four-cycle regimen lasts 42 days. Reasons for a longer cycle duration such as 42 days include, without limitation, the need for a patient to sufficiently recover from the preceding cycle.

Ramp-up dosages of venetoclax on days 1 and 2 of the first cycle can be any dosages below 400 mg that are sound from a therapeutic and safety standpoint. Ramp-up dosages of venetoclax on day 1 of the first cycle include, for example, 25 mg, 50 mg, 75 mg, 100 mg, 125 mg, 150 mg, 175 mg, and 200 mg. Ramp-up dosages of venetoclax on day 2 of the first cycle include, for example, 100 mg, 125 mg, 150 mg, 175 mg, 200 mg, 225 mg, 250 mg, 275 mg, 300 mg, 325 mg, and 350 mg. Ramp-up dosages of venetoclax on days 1 and 2 of the first cycle include, for example, 50 mg and 150 mg, respectively; 75 mg and 150 mg, respectively; 100 mg and 200 mg, respectively; 125 mg and 250 mg, respectively; and 150 mg and 300 mg, respectively. The ramp-up dosages of venetoclax on days 1 and 2 of the first cycle may, for example, be 100 mg and 200 mg, respectively.

The one lintuzumab-Ac225 dose per cycle may, for example, be (i) 0.10 μCi/kg, 0.15 μCi/kg, 0.20 μCi/kg, 0.25 μCi/kg, 0.30 μCi/kg, 0.35 μCi/kg, 0.40 μCi/kg, 0.45 μCi/kg, 0.50 μCi/kg, 0.55 μCi/kg, 0.60 μCi/kg, 0.65 μCi/kg, 0.70 μCi/kg, 0.75 μCi/kg, 0.80 μCi/kg, 0.85 μCi/kg, 0.90 μCi/kg, 0.95 μCi/kg, 1.00 μCi/kg, 1.05 μCi/kg, 1.10 μCi/kg, 1.15 μCi/kg, 1.20 μCi/kg, 1.25 μCi/kg, 1.30 μCi/kg, 1.35 μCi/kg, 1.40 μCi/kg, 1.45 μCi/kg, 1.50 μCi/kg, 1.55 μCi/kg, 1.60 μCi/kg, 1.65 μCi/kg, 1.70 μCi/kg, 1.75 μCi/kg, 1.80 μCi/kg, 1.85 μCi/kg, 1.90 μCi/kg, 1.95 μCi/kg, or 2.00 μCi/kg; (ii) from 0.10 μCi/kg to 0.20 μCi/kg, from 0.20 μCi/kg to 0.30 μCi/kg, from 0.30 μCi/kg to 0.40 μCi/kg, from 0.40 μCi/kg to 0.50 μCi/kg, from 0.50 μCi/kg to 0.60 μCi/kg, from 0.60 μCi/kg to 0.70 μCi/kg, from 0.70 μCi/kg to 0.80 μCi/kg, from 0.80 μCi/kg to 0.90 μCi/kg, from 0.90 μCi/kg to 1.00 μCi/kg, from 1.00 μCi/kg to 1.10 μCi/kg, from 1.10 μCi/kg to 1.20 μCi/kg, from 1.20 μCi/kg to 1.30 μCi/kg, from 1.30 μCi/kg to 1.40 μCi/kg, from 1.40 μCi/kg to 1.50 μCi/kg, from 1.50 μCi/kg to 1.60 μCi/kg, from 1.60 μCi/kg to 1.70 μCi/kg, from 1.70 μCi/kg to 1.80 μCi/kg, from 1.80 μCi/kg to 1.90 μCi/kg, or 1.90 μCi/kg to 2.00 μCi/kg; or (iii) from 0.10 μCi/kg to 0.25 μCi/kg, from 0.25 μCi/kg to 0.50 μCi/kg, from 0.50 μCi/kg to 0.75 μCi/kg, from 0.75 μCi/kg to 1.00 μCi/kg, from 1.00 μCi/kg to 1.25 μCi/kg, from 1.25 μCi/kg to 1.50 μCi/kg, from 1.50 μCi/kg to 1.75 μCi/kg, or from 1.75 μCi/kg to 2.00 μCi/kg. In one embodiment, the lintuzumab-Ac225 dose is 0.25 μCi/kg. In another embodiment, the lintuzumab-Ac225 dose is 0.5 μCi/kg. In a further embodiment, the lintuzumab-Ac225 dose is 1.0 μCi/kg. In yet a further embodiment, the lintuzumab-Ac225 dose is 1.5 μCi/kg. In another embodiment, the lintuzumab-Ac225 is administered (e.g., as an intravenous infusion over 30±10 minutes) on day 4 of at least the first cycle, such as of each cycle. In a still further embodiment, the lintuzumab-Ac225 is administered on day 5 of at least the first cycle, such as of each cycle. In another embodiment, the lintuzumab-Ac225 is administered on day 6 of at least the first cycle, such as of each cycle.

The one lintuzumab-Ac225 dose per cycle may, for example, be the same for all cycles. An example is a regimen including four cycles wherein the lintuzumab-Ac225 dose is 0.5 μCi/kg for each cycle. Alternatively, the one lintuzumab-Ac225 dose per cycle may, for example, not be the same for all cycles, and may, for example, decrease from the first cycle to a subsequent cycles, such as the last cycle. An example is a regimen including four cycles, wherein the lintuzumab-Ac225 dose is 0.5 μCi/kg for each of the first and second cycles, and is 0.25 μCi/kg for each of the third and fourth cycles.

The invention also provides a method for treating a human subject afflicted with acute myeloid leukemia including administering to the subject a regimen of venetoclax and lintuzumab-Ac225, wherein (a) the regimen includes four cycles, each cycle lasting 28 days, (b) the regimen includes (i) orally administering venetoclax at a dose of 100 mg and 200 mg, respectively, on days 1 and 2 of the first cycle, and thereafter orally administering 400 mg of venetoclax daily on days 3-21 of the first cycle and days 1-21 of each subsequent cycle, and (ii) intravenously administering lintuzumab-Ac225 on day 5 of each cycle at a dose of 0.25 μCi/kg, 0.5 μCi/kg, 1.0 μCi/kg, or 1.5 μCi/kg; and (c) the subject has a peripheral blast burden at or below 1,000 blast cells/μl.

In one embodiment of this method, the lintuzumab-Ac225 dose is 0.25 μCi/kg. In another embodiment, the lintuzumab-Ac225 dose is 0.5 μCi/kg. In a further embodiment, the lintuzumab-Ac225 dose is 1.0 μCi/kg. In yet a further embodiment, the lintuzumab-Ac225 dose is 1.5 μCi/kg.

This invention further provides methods employing two administrations of lintuzumab-Ac225 per cycle. For example, the invention provides a method for treating a human subject afflicted with acute myeloid leukemia including administering to the subject a regimen of venetoclax and lintuzumab-Ac225, wherein (a) the regimen includes a plurality of cycles, each cycle lasting from 28 to 60 days, (b) the regimen includes (i) orally administering venetoclax on days 1 and 2 of the first cycle at a ramp-up dosage, and thereafter orally administering 400 mg of venetoclax daily on days 3-21 of the first cycle and days 1-21 of each subsequent cycle, and (ii) intravenously administering lintuzumab-Ac225 on day 4, 5, or 6 of each cycle at a dose of from 0.05 μCi/kg to 1.0 μCi/kg, and on day 18, 19, or 20 of each cycle at a dose of from 0.05 μCi/kg to 1.0 μCi/kg; and (c) the subject has a peripheral blast burden at or below 1,000 blast cells/μl.

In the present method, the regimen may, for example, include two cycles, three cycles, four cycles, five cycles, six cycles, seven cycles, eight cycles, nine cycles, or ten cycles. In one embodiment, the regimen includes four cycles. In another embodiment, the regimen includes eight cycles.

In the present method, each cycle may, for example, last 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, or 60 days. In one embodiment, each cycle lasts 28-30 days, such as 28 days. In another embodiment, not all cycles are of the same duration. In one example, each of the first two cycles of a four-cycle regimen lasts 28 days, and each of the last two cycles of the four-cycle regimen lasts 42 days.

In the present method, the ramp-up dosages of venetoclax on days 1 and 2 of the first cycle can be any dosages below 400 mg that are sound from a therapeutic and safety standpoint. Ramp-up dosages of venetoclax on day 1 of the first cycle include, for example, 25 mg, 50 mg, 75 mg, 100 mg, 125 mg, 150 mg, 175 mg, and 200 mg. Ramp-up dosages of venetoclax on day 2 of the first cycle include, for example, 100 mg, 125 mg, 150 mg, 175 mg, 200 mg, 225 mg, 250 mg, 275 mg, 300 mg, 325 mg, and 350 mg. Ramp-up dosages of venetoclax on days 1 and 2 of the first cycle include, for example, 50 mg and 150 mg, respectively; 75 mg and 150 mg, respectively; 100 mg and 200 mg, respectively; 125 mg and 250 mg, respectively; and 150 mg and 300 mg, respectively. The ramp-up dosages of venetoclax on days 1 and 2 of the first cycle may, for example, be 100 mg and 200 mg, respectively.

In the present method, each of the two lintuzumab-Ac225 doses per cycle may be the same or different and may, for example, be (i) 0.05 μCi/kg, 0.075 μCi/kg, 0.10 μCi/kg, 0.125 μCi/kg, 0.15 μCi/kg, 0.175 μCi/kg, 0.20 μCi/kg, 0.225 μCi/kg, 0.25 μCi/kg, 0.275 Ci/kg, 0.30 μCi/kg, 0.325 μCi/kg, 0.35 μCi/kg, 0.375 μCi/kg, 0.40 μCi/kg, 0.425 μCi/kg, 0.45 μCi/kg, 0.475 μCi/kg, 0.50 μCi/kg, 0.525 μCi/kg, 0.55 μCi/kg, 0.575 μCi/kg, 0.60 μCi/kg, 0.625 μCi/kg, 0.65 μCi/kg, 0.675 μCi/kg, 0.70 μCi/kg, 0.725 μCi/kg, 0.75 μCi/kg, 0.775 μCi/kg, 0.80 μCi/kg, 0.825 μCi/kg, 0.85 μCi/kg, 0.875 μCi/kg, 0.90 μCi/kg, 0.925 μCi/kg, 0.95 μCi/kg, 0.975 μCi/kg, or 1.00 μCi/kg; (ii) from 0.05 μCi/kg to 0.10 μCi/kg, from 0.10 μCi/kg to 0.20 μCi/kg, from 0.20 μCi/kg to 0.30 μCi/kg, from 0.30 μCi/kg to 0.40 μCi/kg, from 0.40 μCi/kg to 0.50 μCi/kg, from 0.50 μCi/kg to 0.60 μCi/kg, from 0.60 μCi/kg to 0.70 μCi/kg, from 0.70 μCi/kg to 0.80 μCi/kg, from 0.80 μCi/kg to 0.90 μCi/kg, or from 0.90 μCi/kg to 1.00 μCi/kg; or (iii) from 0.05 μCi/kg to 0.25 μCi/kg, from 0.25 μCi/kg to 0.50 μCi/kg, from 0.50 μCi/kg to 0.75 μCi/kg, or from 0.75 μCi/kg to 1.00 μCi/kg. In one embodiment, one or both of the lintuzumab-Ac225 doses is 0.125 μCi/kg. In another embodiment, one or both of the lintuzumab-Ac225 doses is 0.25 μCi/kg. In a further embodiment, one or both of the lintuzumab-Ac225 doses is 0.5 μCi/kg. In yet a further embodiment, one or both of the lintuzumab-Ac225 doses is 0.75 μCi/kg. In another embodiment, the lintuzumab-Ac225 is administered (e.g., as an intravenous infusion over 30±10 minutes) on each of the following two days of a cycle, such as the first cycle, such as each cycle: (i) 4 and 18; (ii) 4 and 19; (iii) 4 and 20; (iv) 5 and 18; (v) 5 and 19; (vi) 5 and 20; (vii) 6 and 18; (viii) 6 and 19; or (ix) 6 and 20.

In one embodiment of the present method, the two lintuzumab-Ac225 doses per cycle are the same for all cycles. An example is a regimen including four cycles wherein the twice-administered lintuzumab-Ac225 dose is 0.25 μCi/kg for each cycle. In another embodiment of the present method, the two lintuzumab-Ac225 doses per cycle are not the same for all cycles. For example, the two lintuzumab-Ac225 doses may decrease from the first cycle to a subsequent cycle, such as the last cycle. An example is a regimen including four cycles, wherein the twice-administered lintuzumab-Ac225 dose is 0.25 μCi/kg for each of the first and second cycles, and is 0.125 μCi/kg for each of the third and fourth cycles.

The invention also provides a method for treating a human subject afflicted with acute myeloid leukemia including administering to the subject a regimen of venetoclax and lintuzumab-Ac225, wherein (a) the regimen includes four cycles, each cycle lasting 28 days, (b) the regimen includes (i) orally administering venetoclax at a dose of 100 mg and 200 mg, respectively, on days 1 and 2 of the first cycle, and thereafter orally administering 400 mg of venetoclax daily on days 3-21 of the first cycle and days 1-21 of each subsequent cycle, and (ii) intravenously administering lintuzumab-Ac225 on day 5 of each cycle at a dose of 0.125 μCi/kg, 0.25 μCi/kg, 0.5 μCi/kg, or 0.75 μCi/kg, and on day 19 of each cycle at a dose of 0.125 μCi/kg, 0.25 μCi/kg, 0.5 μCi/kg, or 0.75 μCi/kg; and (c) the subject has a peripheral blast burden at or below 1,000 blast cells/μl.

In one embodiment of this method, the lintuzumab-Ac225 dose is 0.125 μCi/kg. In another embodiment, the lintuzumab-Ac225 dose is 0.25 μCi/kg. In a further embodiment, the lintuzumab-Ac225 dose is 0.5 μCi/kg. In yet a further embodiment, the lintuzumab-Ac225 dose is 0.75 μCi/kg.

In any of the embodiments set forth herein, the subject may, for example, have a peripheral blast burden of (i) below 10 blast cells/μl, below 20 blast cells/μl, below 30 blast cells/μl, below 40 blast cells/μl, below 50 blast cells/μl, below 75 blast cells/μl, below 100 blast cells/μl, below 125 blast cells/μl, below 150 blast cells/μl, below 175 blast cells/μl, below 200 blast cells/μl, below 225 blast cells/μl, below 250 blast cells/μl, below 275 blast cells/μl, below 300 blast cells/μl, below 325 blast cells/μl, below 350 blast cells/μl, below 375 blast cells/μl, below 400 blast cells/μl, below 425 blast cells/μl, below 450 blast cells/μl, below 475 blast cells/μl, below 500 blast cells/μl, below 525 blast cells/μl, below 550 blast cells/μl, below 575 blast cells/μl, below 600 blast cells/μl, below 625 blast cells/μl, below 650 blast cells/μl, below 675 blast cells/μl, below 700 blast cells/μl, below 725 blast cells/μl, below 750 blast cells/μl, below 775 blast cells/μl, below 800 blast cells/μl, below 825 blast cells/μl, below 850 blast cells/μl, below 875 blast cells/μl, below 900 blast cells/μl, below 925 blast cells/μl, below 950 blast cells/μl, below 975 blast cells/μl, or below 1,000 blast cells/μl; (ii) from below 10 or 10 blast cells/μl to 50 blast cells/μl, from 50 blast cells/μl to 100 blast cells/μl, from 100 blast cells/μl to 150 blast cells/μl, from 150 blast cells/μl to 200 blast cells/μl, from 200 blast cells/μl to 250 blast cells/μl, from 250 blast cells/μl to 300 blast cells/μl, from 300 blast cells/μl to 350 blast cells/μl, from 350 blast cells/μl to 400 blast cells/μl, from 400 blast cells/μl to 450 blast cells/μl, from 450 blast cells/μl to 500 blast cells/μl, from 500 blast cells/μl to 550 blast cells/μl, from 550 blast cells/μl to 600 blast cells/μl, from 600 blast cells/μl to 650 blast cells/μl, from 650 blast cells/μl to 700 blast cells/μl, from 700 blast cells/μl to 750 blast cells/μl, from 750 blast cells/μl to 800 blast cells/μl, from 800 blast cells/μl to 850 blast cells/μl, from 850 blast cells/μl to 900 blast cells/μl, from 900 blast cells/μl to 950 blast cells/μl, or from 950 blast cells/μl to 1,000 blast cells/μl; or (iii) from below 10 or 10 blast cells/μl to 100 blast cells/μl, from 100 blast cells/μl to 200 blast cells/μl, from 200 blast cells/μl to 300 blast cells/μl, from 300 blast cells/μl to 400 blast cells/μl, from 400 blast cells/μl to 500 blast cells/μl, from 500 blast cells/μl to 600 blast cells/μl, from 600 blast cells/μl to 700 blast cells/μl, from 700 blast cells/μl to 800 blast cells/μl, from 800 blast cells/μl to 900 blast cells/μl, or from 900 blast cells/μl to 1,000 blast cells/μl.

Without limitation, the following enumerated embodiments of the invention are also provided:

Embodiment 1. A method for treating a human subject afflicted with acute myeloid leukemia including administering to the subject a regimen of venetoclax and lintuzumab-Ac225, wherein (a) the regimen includes a plurality of cycles, each cycle lasting from 28 to 60 days, (b) the regimen includes (i) orally administering venetoclax on days 1 and 2 of the first cycle at a ramp-up dosage, and thereafter orally administering 400 mg of venetoclax daily on days 3-21 of the first cycle and days 1-21 of each subsequent cycle, and (ii) intravenously administering lintuzumab-Ac225 on day 4, 5, 6 or 7 of each cycle at a dose of from 0.1 μCi/kg to 2.0 μCi/kg; and (c) the subject has a peripheral blast burden at or below 1,000 blast cells/μl.

Embodiment 2. The method of embodiment 1, wherein the regimen includes four cycles.

Embodiment 3. The method of embodiment 1, wherein the regimen includes eight cycles.

Embodiment 4. The method of any one of embodiments 1-3, wherein each cycle lasts 28 days.

Embodiment 5. The method of any one of embodiments 1-4, wherein the ramp-up dosages of venetoclax on days 1 and 2 of the first cycle are 100 mg and 200 mg, respectively.

Embodiment 6. The method of any one of embodiments 1-5, wherein the lintuzumab-Ac225 dose is 0.25 μCi/kg, 0.5 μCi/kg, 1.0 μCi/kg, or 1.5 μCi/kg.

Embodiment 7. A method for treating a human subject afflicted with acute myeloid leukemia including administering to the subject a regimen of venetoclax and lintuzumab-Ac225, wherein (a) the regimen includes four cycles, each cycle lasting 28 days, (b) the regimen includes (i) orally administering venetoclax at a dose of 100 mg and 200 mg, respectively, on days 1 and 2 of the first cycle, and thereafter orally administering 400 mg of venetoclax daily on days 3-21 of the first cycle and days 1-21 of each subsequent cycle, and (ii) intravenously administering lintuzumab-Ac225 on day 5 of each cycle at a dose of 0.25 μCi/kg, 0.5 μCi/kg, 1.0 μCi/kg, or 1.5 μCi/kg; and (c) the subject has a peripheral blast burden at or below 1,000 blast cells/μl.

Embodiment 8. A method for treating a human subject afflicted with acute myeloid leukemia including administering to the subject a regimen of venetoclax and lintuzumab-Ac225, wherein (a) the regimen includes a plurality of cycles, each cycle lasting from 28 to 60 days, (b) the regimen includes (i) orally administering venetoclax on days 1 and 2 of the first cycle at a ramp-up dosage, and thereafter orally administering 400 mg of venetoclax daily on days 3-21 of the first cycle and days 1-21 of each subsequent cycle, and (ii) intravenously administering lintuzumab-Ac225 on day 4, 5, 6 or 7 of each cycle at a dose of from 0.05 μCi/kg to 1.0 μCi/kg, and on day 18, 19, 20 or 21 of each cycle at a dose of from 0.05 μCi/kg to 1.0 μCi/kg; and (c) the subject has a peripheral blast burden at or below 1,000 blast cells/μl.

Embodiment 9. The method of embodiment 8, wherein the regimen includes four cycles.

Embodiment 10. The method of embodiment 8, wherein the regimen includes eight cycles.

Embodiment 11. The method of any one of embodiments 8-10, wherein each cycle lasts 28-45 days or any number of days in said range, such as 28 days.

Embodiment 12. The method of any one of embodiments 8-11, wherein the ramp-up dosages of venetoclax on days 1 and 2 of the first cycle are 100 mg and 200 mg, respectively.

Embodiment 13. The method of any one of embodiments 8-12, wherein the lintuzumab-Ac225 dose is 0.125 μCi/kg, 0.25 μCi/kg, 0.5 μCi/kg, or 0.75 μCi/kg.

Embodiment 14. A method for treating a human subject afflicted with acute myeloid leukemia including administering to the subject a regimen of venetoclax and lintuzumab-Ac225, wherein (a) the regimen includes four cycles, each cycle lasting 28 days, (b) the regimen includes (i) orally administering venetoclax at a dose of 100 mg and 200 mg, respectively, on days 1 and 2 of the first cycle, and thereafter orally administering 400 mg of venetoclax daily on days 3-21 of the first cycle and days 1-21 of each subsequent cycle, and (ii) intravenously administering lintuzumab-Ac225 on day 5 of each cycle at a dose of 0.125 μCi/kg, 0.25 μCi/kg, 0.5 μCi/kg, or 0.75 μCi/kg, and on day 19 of each cycle at a dose of 0.125 μCi/kg, 0.25 μCi/kg, 0.5 μCi/kg, or 0.75 μCi/kg; and (c) the subject has a peripheral blast burden at or below 1,000 blast cells/μl.

Embodiment 15. Combination use of lintuzumab-Ac225 and venetoclax for the treatment of AML in a human subject in a treatment regimen in which daily administration, such as oral administration, of venetoclax begins on day 1 and administration, such as intravenous administration, of lintuzumab-Ac225 occurs on day 4, 5, 6 or 7.

Embodiment 16. The combination use of embodiment 15, wherein the treatment regimen includes one or more cycles lasting 28-60 days or any number of days in said range, such as at least two such cycles.

Embodiment 17. The combination use of embodiment 15 or 16, wherein in the treatment regimen the dose of lintuzumab-Ac225 administered on day 4, 5, 6, or 7 is in the range of 0.05 μCi/kg to 1.0 μCi/kg or the range of 0.1 μCi/kg to 2.0 μCi/kg, such as 0.125 μCi/kg, 0.25 μCi/kg, 0.5 μCi/kg, 0.75 μCi/kg, 1.0 μCi/kg or 1.5 μCi/kg.

Embodiment 18. The combination use of any one of embodiments 15-17, wherein in the treatment regimen venetoclax is administered on days 1 and 2 (such as of a first cycle) at a ramp-up dosage, such as 100 mg on day 1 and 200 mg on day 2, and thereafter administered at 400 mg daily, such as on days 3-21, such as on days 3-21 of a first cycle.

Embodiment 19. The combination use of embodiment 18, wherein in the treatment regimen venetoclax is administered at 400 mg daily on days 1-21 of any subsequent cycle (i.e., cycle 2 and any cycles thereafter).

Embodiment 20. The combination use of any one of embodiments 15-19, wherein in the treatment regimen lintuzumab-Ac225 is administered on day 17, 18, 19, 20 or 21 such as at least on day 17, 18, 19, 20 or 21 of at least a first cycle, such as the first cycle and any subsequent cycle.

Embodiment 21. The combination use of embodiment 20, wherein in the treatment regimen the dose of lintuzumab-Ac225 administered on day 17, 18, 19, 20 or 21 is in the range of 0.05 μCi/kg to 1.0 μCi/kg or the range of 0.1 μCi/kg to 2.0 μCi/kg, such as 0.125 μCi/kg, 0.25 μCi/kg, 0.5 μCi/kg, 0.75 μCi/kg, 1.0 μCi/kg or 1.5 μCi/kg.

Embodiment 22. The combination use of any one of embodiments 15-21, wherein the use is for the treatment of AML in a human subject having a peripheral blast burden at or below 1,000 blast cells/μl.

Embodiment 23. A pharmaceutical composition comprising lintuzumab-Ac225 for the treatment of AML in a human subject in combination with venetoclax in a treatment regimen in which daily administration, such as oral administration, of venetoclax begins on day 1 and administration, such as intravenous administration, of lintuzumab-Ac225 occurs on day 4, 5, 6 or 7.

Embodiment 24. The pharmaceutical composition of embodiment 23, wherein the treatment regimen includes one or more cycles lasting 28-60 days or any number of days in said range, such as at least two such cycles.

Embodiment 25. The pharmaceutical composition of embodiment 23 or 24, wherein in the treatment regimen the dose of lintuzumab-Ac225 administered on day 4, 5, 6, or 7 is in the range of 0.05 μCi/kg to 1.0 μCi/kg or the range of 0.1 μCi/kg to 2.0 μCi/kg, such as 0.125 μCi/kg, 0.25 μCi/kg, 0.5 μCi/kg, 0.75 μCi/kg, 1.0 μCi/kg or 1.5 μCi/kg.

Embodiment 26. The pharmaceutical composition of any one of embodiments 23-25, wherein in the treatment regimen venetoclax is administered on days 1 and 2 of the first cycle at a ramp-up dosage, such as 100 mg on day 1 and 200 mg on day 2, and thereafter administered at 400 mg daily on days 3-21 of a first cycle.

Embodiment 27. The pharmaceutical composition of embodiment 26, wherein in the treatment regimen venetoclax is administered at 400 mg daily on days 1-21 of any subsequent cycle (i.e., cycle 2 and any cycles thereafter).

Embodiment 28. The pharmaceutical composition of any one of embodiments 23-27, wherein in the treatment regimen lintuzumab-Ac225 is administered on day 17, 18, 19, 20 or 21 such as at least on day 17, 18, 19, 20 or 21 of at least a first cycle, such as the first cycle and any subsequent cycle.

Embodiment 29. The pharmaceutical composition of embodiment 28, wherein in the treatment regimen the dose of lintuzumab-Ac225 administered on day 17, 18, 19, 20 or 21 is in the range of 0.05 μCi/kg to 1.0 μCi/kg or the range of 0.1 μCi/kg to 2.0 μCi/kg, such as 0.125 μCi/kg, 0.25 μCi/kg, 0.5 μCi/kg, 0.75 μCi/kg, 1.0 μCi/kg or 1.5 μCi/kg.

Embodiment 30. The pharmaceutical composition of any one of embodiments 23-29, wherein the composition is for the treatment of AML in a human subject having a peripheral blast burden at or below 1,000 blast cells/μl.

Embodiment 31. The pharmaceutical composition of any one of embodiments 23-30, further comprising at least one pharmaceutically acceptable excipient.

Embodiment 32. A pharmaceutical composition comprising venetoclax for the treatment of AML in a human subject in combination with lintuzumab-Ac225 in a treatment regimen in which daily administration, such as oral administration, of venetoclax begins on day 1 and administration, such as intravenous administration, of lintuzumab-Ac225 occurs on day 4, 5, 6 or 7.

Embodiment 33. The pharmaceutical composition of embodiment 32, wherein the treatment regimen includes one or more cycles lasting 28-60 days or any number of days in said range, such as at least two such cycles.

Embodiment 34. The pharmaceutical composition of embodiment 32 or 33, wherein in the treatment regimen the dose of lintuzumab-Ac225 administered on day 4, 5, 6, or 7 is in the range of 0.05 μCi/kg to 1.0 μCi/kg or the range of 0.1 μCi/kg to 2.0 μCi/kg, such as 0.125 μCi/kg, 0.25 μCi/kg, 0.5 μCi/kg, 0.75 μCi/kg, 1.0 μCi/kg or 1.5 μCi/kg.

Embodiment 35. The pharmaceutical composition of any one of embodiments 32-34, wherein in the treatment regimen venetoclax is administered on days 1 and 2 of the first cycle at a ramp-up dosage, such as 100 mg on day 1 and 200 mg on day 2, and thereafter administered at 400 mg daily on days 3-21 of a first cycle.

Embodiment 36. The pharmaceutical composition of embodiment 35, wherein in the treatment regimen venetoclax is administered at 400 mg daily on days 1-21 of any subsequent cycle (i.e., cycle 2 and any cycles thereafter).

Embodiment 37. The pharmaceutical composition of any one of embodiments 32-36, wherein in the treatment regimen lintuzumab-Ac225 is administered on day 17, 18, 19, 20 or 21 such as at least on day 17, 18, 19, 20 or 21 of at least a first cycle, such as the first cycle and any subsequent cycle.

Embodiment 38. The pharmaceutical composition of embodiment 37, wherein in the treatment regimen the dose of lintuzumab-Ac225 administered on day 17, 18, 19, 20 or 21 is in the range of 0.05 μCi/kg to 1.0 μCi/kg or the range of 0.1 μCi/kg to 2.0 μCi/kg, such as 0.125 μCi/kg, 0.25 μCi/kg, 0.5 μCi/kg, 0.75 μCi/kg, 1.0 μCi/kg or 1.5 μCi/kg.

Embodiment 39. The pharmaceutical composition of any one of embodiments 32-38, wherein the composition is for the treatment of AML in a human subject having a peripheral blast burden at or below 1,000 blast cells/μl.

Embodiment 40. The pharmaceutical composition of any one of embodiments 32-39, further comprising at least one pharmaceutically acceptable excipient.

It should be understood that wherever in this disclosure various methods involving the administration of lintuzumab-Ac225 and venetoclax according to various regimens are described, corresponding combination use embodiments in accordance with the same regimens for the treatment of AML and corresponding pharmaceutical composition embodiments for the treatment of AML according to the same regimens are also intended to be disclosed and are provided by the present invention.

Without limitation, various aspects and embodiments of the invention are further illustrated with reference to the following examples.

### Example 1—Structure of 225Ac-Lintuzumab (Lintuzumab-Ac225; 225 Ac-HuM195)

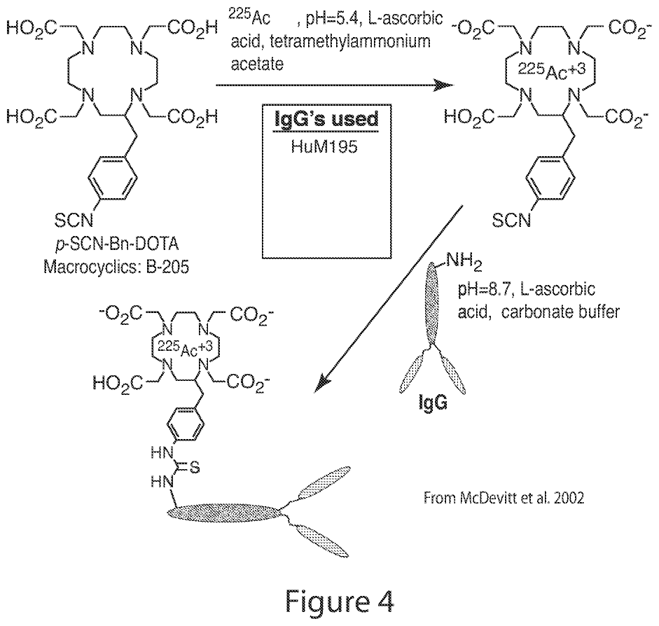

²²⁵Ac-lintuzumab includes three key components; humanized monoclonal antibody HuM195, the alpha-emitting radioisotope ²²⁵Ac, and the bi-functional chelate/chelator 2-(p-isothiocyanatobenzyl)-1,4,7,10-tetraazacyclododecane-1,4,7,10-tetraacetic acid (p-SCN-Bn-DOTA). As depicted in FIG. 4, HuM195 is radiolabeled using the bi-functional chelate/chelator p-SCN-Bn-DOTA that binds to ²²⁵Ac and that is covalently attached to the IgG via a lysine residue on the antibody.

### Example 2—p-SCN-Bn-DOTA

DOTA, 2-(4-Isothiocyanatobenzyl)-1,4,7,10-tetraazacyclododecane tetraacetic acid (Macrocyclics item code B205-GMP) is synthesized by a multi-step organic synthesis that is fully described in U.S. Pat. No. 4,923,985.

### Example 3—Preparation of ²²⁵Ac-Lintuzumab (²²⁵Ac-HuM195)

The procedure for preparing ²²⁵Ac-lintuzumab is based on the method described by Michael R. McDevitt, “Design and synthesis of ²²⁵Ac radioimmuno-pharmaceuticals, Applied Radiation and Isotope”, 57 (2002), 841-847. The procedure involves radiolabeling p-SCN-Bn-DOTA, with the radioisotope ²²⁵Ac, followed by chemical conjugation of the radiolabeled p-SCN-Bn-DOTA to the antibody (HuM195). The construct, ²²⁵Ac-p-SCN-Bn-DOTA-HuM195, is purified using 10 DG size exclusion chromatography and eluted with 1% human serum albumin (HSA). The resulting drug product, ²²⁵Ac-lintuzumab, is then passed through a 0.2 μm sterilizing filter.

### Example 4—Process Flow for Preparation of ²²⁵Ac-Lintuzumab (225Ac-HuM195)

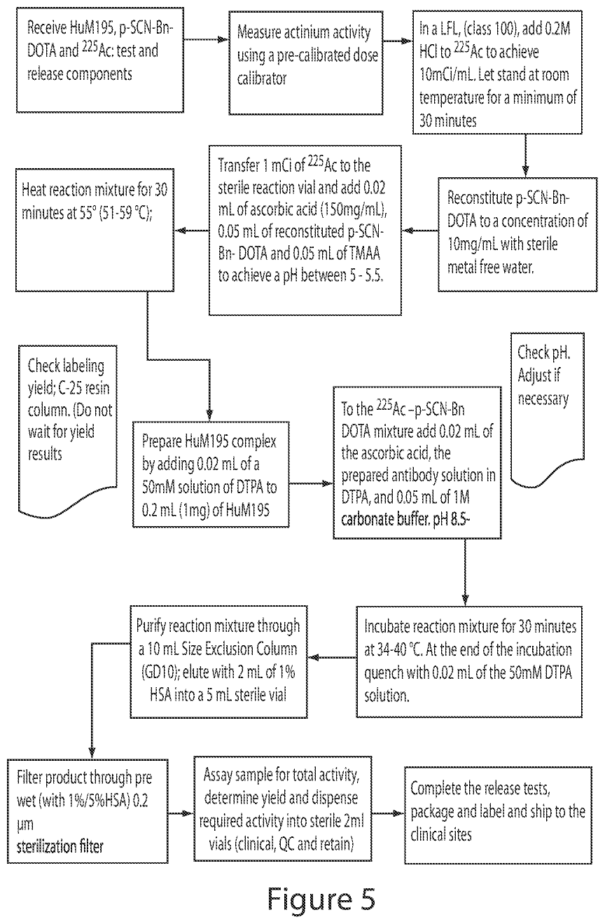

The procedure, shown in FIG. 5, begins with confirming the identity of all components and the subsequent QC release of the components to production. The ²²⁵Ac is assayed to confirm the level of activity and is reconstituted to the desired activity concentration with hydrochloric acid. A vial of lyophilized p-SCN-Bn-DOTA is reconstituted with metal-free water to a concentration of 10 mg/ml. To the actinium reaction vial, 0.02 ml of ascorbic acid solution (150 mg/ml) and 0.05 ml of reconstituted p-SCN-Bn-DOTA are added and the pH adjusted to between 5 and 5.5 with 2M tetramethylammonium acetate (TMAA). The mixture is then heated at 55±4° C. for 30 minutes.

To determine the labeling efficiency of the ²²⁵Ac-p-SCN-Bn-DOTA, an aliquot of the reaction mixture is removed and applied to a 1 ml column of Sephadex C25 cation exchange resin. The product is eluted in 2-4 ml fractions with a 0.9% saline solution. The fraction of ²²⁵Ac activity that elutes is ²²⁵Ac-p-SCN-Bn-DOTA and the fraction that is retained on the column is un-chelated, unreactive 225Ac. Typically, the labeling efficiency is greater than 95%.

To the reaction mixture, 0.22 ml of previously prepared HuM195 in DTPA (1 mg HuM195) and 0.02 ml of ascorbic acid are added. The DTPA is added to bind any trace amounts of metals that may compete with the labeling of the antibody. The ascorbic acid is added as a radio-protectant. The pH is adjusted with carbonate buffer to pH 8.5-9. The mixture is heated at 37±3° C. for 30 minutes.

The final product is purified by size exclusion chromatography using 10DG resin and eluted with 2 ml of 1% HSA. Typical reaction yields are 10%.

### Example 5—Venetoclax and its Normal Dosing Regimen

Venetoclax is sold by Genentech (San Francisco, CA) under the brand name Venclexta™. According to the FDA's Venclexta™ label, this drug “is a BCL-2 inhibitor indicated for the treatment of patients with chronic lymphocytic leukemia (CLL) with 17p deletion who have received at least one prior therapy.” Venclexta™ is sold in tablet form at 10 mg, 50 mg and 100 mg. Therapy is to be initiated “at 20 mg once daily for 7 days, followed by a weekly ramp-up dosing schedule to the recommended daily dose of 400 mg.” The ramp-up dosing schedule is as follows: week 1, 20 mg/day; week 2, 50 mg/day; week 3, 100 mg/day; week 4, 200 mg/day; and week 5 and beyond, 400 mg/day.

### Example 6—²²⁵Ac-Lintuzumab and Venetoclax—Scenario I

In this scenario for treating a human subject afflicted with acute myeloid leukemia, the present method includes administering to the subject a regimen of venetoclax and lintuzumab-Ac225, wherein (a) the regimen includes four cycles, each cycle lasting 28 days, (b) the regimen includes (i) orally administering venetoclax at a dose of 100 mg and 200 mg, respectively, on days 1 and 2 of the first cycle, and thereafter orally administering 400 mg of venetoclax daily on days 3-21 of the first cycle and days 1-21 of each subsequent cycle, and (ii) intravenously administering lintuzumab-Ac225 on day 5 of each cycle at a dose of 0.25 μCi/kg; and (c) the subject has a peripheral blast burden at or below 1,000 blast cells/μl. In one variation, the method includes intravenously administering lintuzumab-Ac225 on day 4 of each cycle at a dose of 0.25 μCi/kg. In another variation, the method includes intravenously administering lintuzumab-Ac225 on day 6 of each cycle at a dose of 0.25 μCi/kg.

### Example 7—²²⁵Ac-Lintuzumab and Venetoclax—Scenario II

In this scenario for treating a human subject afflicted with acute myeloid leukemia, the present method includes administering to the subject a regimen of venetoclax and lintuzumab-Ac225, wherein (a) the regimen includes four cycles, each cycle lasting 28 days, (b) the regimen includes (i) orally administering venetoclax at a dose of 100 mg and 200 mg, respectively, on days 1 and 2 of the first cycle, and thereafter orally administering 400 mg of venetoclax daily on days 3-21 of the first cycle and days 1-21 of each subsequent cycle, and (ii) intravenously administering lintuzumab-Ac225 on day 5 of each cycle at a dose of 0.5 μCi/kg; and (c) the subject has a peripheral blast burden at or below 1,000 blast cells/μl. In one variation, the method includes intravenously administering lintuzumab-Ac225 on day 4 of each cycle at a dose of 0.5 μCi/kg. In another variation, the method includes intravenously administering lintuzumab-Ac225 on day 6 of each cycle at a dose of 0.5 μCi/kg.

### Example 8—225Ac-Lintuzumab and Venetoclax—Scenario III

In this scenario for treating a human subject afflicted with acute myeloid leukemia, the present method includes administering to the subject a regimen of venetoclax and lintuzumab-Ac225, wherein (a) the regimen includes four cycles, each cycle lasting 28 days, (b) the regimen includes (i) orally administering venetoclax at a dose of 100 mg and 200 mg, respectively, on days 1 and 2 of the first cycle, and thereafter orally administering 400 mg of venetoclax daily on days 3-21 of the first cycle and days 1-21 of each subsequent cycle, and (ii) intravenously administering lintuzumab-Ac225 on day 5 of each cycle at a dose of 1.0 μCi/kg; and (c) the subject has a peripheral blast burden at or below 1,000 blast cells/μl. In one variation, the method includes intravenously administering lintuzumab-Ac225 on day 4 of each cycle at a dose of 1.0 μCi/kg. In another variation, the method includes intravenously administering lintuzumab-Ac225 on day 6 of each cycle at a dose of 1.0 μCi/kg.

### Example 9—²²⁵Ac-Lintuzumab and Venetoclax—Scenario IV

In this scenario for treating a human subject afflicted with acute myeloid leukemia, the present method includes administering to the subject a regimen of venetoclax and lintuzumab-Ac225, wherein (a) the regimen includes four cycles, each cycle lasting 28 days, (b) the regimen includes (i) orally administering venetoclax at a dose of 100 mg and 200 mg, respectively, on days 1 and 2 of the first cycle, and thereafter orally administering 400 mg of venetoclax daily on days 3-21 of the first cycle and days 1-21 of each subsequent cycle, and (ii) intravenously administering lintuzumab-Ac225 on day 5 of each cycle at a dose of 1.5 μCi/kg; and (c) the subject has a peripheral blast burden at or below 1,000 blast cells/μl. In one variation, the method includes intravenously administering lintuzumab-Ac225 on day 4 of each cycle at a dose of 1.5 μCi/kg. In another variation, the method includes intravenously administering lintuzumab-Ac225 on day 6 of each cycle at a dose of 1.5 μCi/kg.

### Example 10—²²⁵Ac-Lintuzumab and Venetoclax—Scenario V

In this scenario for treating a human subject afflicted with acute myeloid leukemia, the present method includes administering to the subject a regimen of venetoclax and lintuzumab-Ac225, wherein (a) the regimen includes four cycles, each cycle lasting 28 days, (b) the regimen includes (i) orally administering venetoclax at a dose of 100 mg and 200 mg, respectively, on days 1 and 2 of the first cycle, and thereafter orally administering 400 mg of venetoclax daily on days 3-21 of the first cycle and days 1-21 of each subsequent cycle, and (ii) intravenously administering lintuzumab-Ac225 on day 5 of each cycle at a dose of 0.125 μCi/kg, and on day 19 of each cycle at a dose of 0.125 μCi/kg; and (c) the subject has a peripheral blast burden at or below 1,000 blast cells/μl. In one variation, the method includes intravenously administering lintuzumab-Ac225 at a dose of 0.125 μCi/kg on each of days 4 and 18 of each cycle. In another further variation, the method includes intravenously administering lintuzumab-Ac225 at a dose of 0.125 μCi/kg on each of days 4 and 19 of each cycle. In a further variation, the method includes intravenously administering lintuzumab-Ac225 at a dose of 0.125 μCi/kg on each of days 4 and 20 of each cycle. In a still further variation, the method includes intravenously administering lintuzumab-Ac225 at a dose of 0.125 μCi/kg on each of days 5 and 18 of each cycle. In another variation, the method includes intravenously administering lintuzumab-Ac225 at a dose of 0.125 μCi/kg on each of days 5 and 20 of each cycle. In a further variation, the method includes intravenously administering lintuzumab-Ac225 at a dose of 0.125 μCi/kg on each of days 6 and 18 of each cycle. In a still further variation, the method includes intravenously administering lintuzumab-Ac225 at a dose of 0.125 μCi/kg on each of days 6 and 19 of each cycle. In another variation, the method includes intravenously administering lintuzumab-Ac225 at a dose of 0.125 μCi/kg on each of days 6 and 20 of each cycle.

### Example 11—²²⁵Ac-Lintuzumab and Venetoclax—Scenario VI

In this scenario for treating a human subject afflicted with acute myeloid leukemia, the present method includes administering to the subject a regimen of venetoclax and lintuzumab-Ac225, wherein (a) the regimen includes four cycles, each cycle lasting 28 days, (b) the regimen includes (i) orally administering venetoclax at a dose of 100 mg and 200 mg, respectively, on days 1 and 2 of the first cycle, and thereafter orally administering 400 mg of venetoclax daily on days 3-21 of the first cycle and days 1-21 of each subsequent cycle, and (ii) intravenously administering lintuzumab-Ac225 on day 5 of each cycle at a dose of 0.25 μCi/kg, and on day 19 of each cycle at a dose of 0.25 μCi/kg; and (c) the subject has a peripheral blast burden at or below 1,000 blast cells/μl. In one variation, the method includes intravenously administering lintuzumab-Ac225 at a dose of 0.25 μCi/kg on each of days 4 and 18 of each cycle. In another variation, the method includes intravenously administering lintuzumab-Ac225 at a dose of 0.25 μCi/kg on each of days 4 and 19 of each cycle. In still another variation, the method includes intravenously administering lintuzumab-Ac225 at a dose of 0.25 μCi/kg on each of days 4 and 20 of each cycle. In a further variation, the method includes intravenously administering lintuzumab-Ac225 at a dose of 0.25 μCi/kg on each of days 5 and 18 of each cycle. In a still further variation, the method includes intravenously administering lintuzumab-Ac225 at a dose of 0.25 μCi/kg on each of days 5 and 20 of each cycle. In another variation, the method includes intravenously administering lintuzumab-Ac225 at a dose of 0.25 Ci/kg on each of days 6 and 18 of each cycle. In still another variation, the method includes intravenously administering lintuzumab-Ac225 at a dose of 0.25 μCi/kg on each of days 6 and 19 of each cycle. In a further variation, the method includes intravenously administering lintuzumab-Ac225 at a dose of 0.25 μCi/kg on each of days 6 and 20 of each cycle.

### Example 12—²²⁵Ac-Lintuzumab and Venetoclax—Scenario VII

In this scenario for treating a human subject afflicted with acute myeloid leukemia, the present method includes administering to the subject a regimen of venetoclax and lintuzumab-Ac225, wherein (a) the regimen includes four cycles, each cycle lasting 28 days, (b) the regimen includes (i) orally administering venetoclax at a dose of 100 mg and 200 mg, respectively, on days 1 and 2 of the first cycle, and thereafter orally administering 400 mg of venetoclax daily on days 3-21 of the first cycle and days 1-21 of each subsequent cycle, and (ii) intravenously administering lintuzumab-Ac225 on day 5 of each cycle at a dose of 0.5 μCi/kg, and on day 19 of each cycle at a dose of 0.5 μCi/kg; and (c) the subject has a peripheral blast burden at or below 1,000 blast cells/μl. In one variation, the method includes intravenously administering lintuzumab-Ac225 at a dose of 0.5 μCi/kg on each of days 4 and 18 of each cycle. In another variation, the method includes intravenously administering lintuzumab-Ac225 at a dose of 0.5 μCi/kg on each of days 4 and 19 of each cycle. In still another variation, the method includes intravenously administering lintuzumab-Ac225 at a dose of 0.5 μCi/kg on each of days 4 and 20 of each cycle. In a further variation, the method includes intravenously administering lintuzumab-Ac225 at a dose of 0.5 μCi/kg on each of days 5 and 18 of each cycle. In a still further variation, the method includes intravenously administering lintuzumab-Ac225 at a dose of 0.5 μCi/kg on each of days 5 and 20 of each cycle. In another variation, the method includes intravenously administering lintuzumab-Ac225 at a dose of 0.5 μCi/kg on each of days 6 and 18 of each cycle. In still another variation, the method includes intravenously administering lintuzumab-Ac225 at a dose of 0.5 μCi/kg on each of days 6 and 19 of each cycle. In a further variation, the method includes intravenously administering lintuzumab-Ac225 at a dose of 0.5 μCi/kg on each of days 6 and 20 of each cycle.

### Example 13—²²⁵Ac-Lintuzumab and Venetoclax—Scenario VIII

In this scenario for treating a human subject afflicted with acute myeloid leukemia, the present method includes administering to the subject a regimen of venetoclax and lintuzumab-Ac225, wherein (a) the regimen includes four cycles, each cycle lasting 28 days, (b) the regimen includes (i) orally administering venetoclax at a dose of 100 mg and 200 mg, respectively, on days 1 and 2 of the first cycle, and thereafter orally administering 400 mg of venetoclax daily on days 3-21 of the first cycle and days 1-21 of each subsequent cycle, and (ii) intravenously administering lintuzumab-Ac225 on day 5 of each cycle at a dose of 0.75 μCi/kg, and on day 19 of each cycle at a dose of 0.75 μCi/kg; and (c) the subject has a peripheral blast burden at or below 1,000 blast cells/μl. In one variation, the method includes intravenously administering lintuzumab-Ac225 at a dose of 0.75 μCi/kg on each of days 4 and 18 of each cycle. In another variation, the method includes intravenously administering lintuzumab-Ac225 at a dose of 0.75 μCi/kg on each of days 4 and 19 of each cycle. In still another variation, the method includes intravenously administering lintuzumab-Ac225 at a dose of 0.75 μCi/kg on each of days 4 and 20 of each cycle. In a further variation, the method includes intravenously administering lintuzumab-Ac225 at a dose of 0.75 μCi/kg on each of days 5 and 18 of each cycle. In a still further variation, the method includes intravenously administering lintuzumab-Ac225 at a dose of 0.75 μCi/kg on each of days 5 and 20 of each cycle. In another variation, the method includes intravenously administering lintuzumab-Ac225 at a dose of 0.75 μCi/kg on each of days 6 and 18 of each cycle. In still another variation, the method includes intravenously administering lintuzumab-Ac225 at a dose of 0.75 μCi/kg on each of days 6 and 19 of each cycle. In a further variation, the method includes intravenously administering lintuzumab-Ac225 at a dose of 0.75 μCi/kg on each of days 6 and 20 of each cycle.

### Example 14—Extension of Cycle Duration

Each cycle of a treatment regimen may be designated as 28 days at its outset, but in the event of neutropenia or thrombocytopenia (Grade 4), the cycle length can be extended (e.g., to 42 days) to allow for recovery of cell counts. A bone marrow biopsy/aspirate, completed at cycle 1, day 28 (+3 days), can inform the decision to extend to 42 days versus proceeding directly to cycle 2. If there is persistent disease with evidence of response (below 5% blasts in bone marrow), proceeding to cycle 2 without additional delay is preferred.

### Example 15—Supportive Care

In one variation of the present methods, the patient receives spironolactone (at a daily oral dose of 25 mg) starting on day 15 of cycle 1 until one year after the last lintuzumab-Ac225 administration, unless clinically contraindicated. Eplerenone may be used for those who are allergic to or intolerant of spironolactone. In another variation, hydroxyurea is administered prior to cycle 1 (and during cycles 1 and 2, if clinically needed) to control leukemic proliferation. In still another variation, anti-hyperuricemic agents, such as allopurinol, are administered starting at least three days prior to cycle 1 and during the first week of treatment with venetoclax as a prophylaxis for tumor lysis syndrome.

### Example 16—Hydroxyurea and Hydration

In one variation of the present methods, treatment with hydroxyurea prior to and during cycle 1 is maximized to lower circulating blast counts to below 1000/μl prior to day 5, when lintuzumab-Ac225 is administered. Hydroxyurea is used when necessary to lower circulating blast counts during screening and during cycle 1 and cycle 2.

In another variation of the present methods, the patient receives 750 ml intravenous hydration over at least three hours before infusion of lintuzumab-Ac225 therapy and at least 500 ml over at least three hours post infusion. The patient ideally maintains a urine output of at least 75 ml/hour, on average, during the pre- and post-hydration period. Additionally, acetaminophen (325-650 mg oral) and diphenhydramine (25-50 mg oral or intravenous) may be given 30-60 minutes prior to the infusion start to prevent infusion-related reactions.

### Example 17—Day 1 Versus Day 5 Administration of ²²⁵Ac-Lintuzumab in Combination with Venetoclax in Relapsed/Refractory AML Patients

A Phase I clinical study employing a 3+3 dose-escalation design was undertaken to determine the maximum tolerated dose (MTD) of lintuzumab-225Ac when given in combination with venetoclax to R/R AML patients. The planned dose levels for lintuzumab-Ac225 were 0.5, 1.0, and 1.5 μCi/kg. This example relates to the first cohort of three patients to receive 0.5 μCi/kg lintuzumab-Ac225 in this study.

Three initial R/R AML patients with a median age of 54 years (range 49-75) were enrolled (see patient profiles on Table 1). These patients had a median of 2 prior therapies (2-3) and a median bone marrow blast percentage of 30% (range 20->60). All 3 patients were poor risk with adverse cytogenetics as well as an additional high-risk marker (FLT3-ITD+, antecedent JAK2+ myelofibrosis, or TP53 mutation).

Treatment cycles were 28 to 42 days long. The patients received hydroxyurea prior to cycle 1 through cycle 2 as needed, anti-hyperuricemia agents at least Day −3 through Day 7, and venetoclax at 400 mg/day PO on Days 1 to 21 of each cycle with a cycle 1 dose ramp-up to reduce the risk of tumor lysis syndrome. Patients 1 and 2 were administered 0.5 μCi/kg lintuzumab-Ac225 as a single dose on Day 1 of each cycle and Patient 3 was administered 0.5 μCi/kg lintuzumab-Ac225 as a single dose on Day 5 of each cycle.

TABLE 1

| Patient No. | 1 | 2 | 3 |
|---|---|---|---|
| Age | 54 | 49 | 75 |
| Sex | M | M | M |
| # prior therapies | 3 | 2 | 2 |
| Relapse/refractory | Refractory | Relapsed | 2nd Relapse |
| BM % Blasts | >60% | 30% | 20-25% |
| BM % CD33+ | 100% | 100% | 30% |
| Cytogenetics | Adverse | Adverse | Adverse |
| Molecular analysis | FLT3-ITD | JAK2, SRSF2 | ASXL1, BRAF, |
| (+) | | | IDH2, RUNX1, |
| | | | SRSF2, TP53 |
| Antecedent | N/A | Myelofibrosis | N/A |
| MDS/MPN | | | |

### Results:

No lintuzumab-Ac225 related dose limiting toxicities (DLT) or non-hematologic Grade 3 or greater related AEs were observed in these patients.

Patient 1, who received lintuzumab-Ac225 on Day 1, achieved a partial response with a decrease in BM leukemic blasts from >60% to 30% seen on Day 28. However, despite the partial response seen in the Day 28 BM, the patient had persistence of FLT3-ITD and increasing % of peripheral blood blasts and was discontinued from the study.

Patient 2, who received lintuzumab-Ac225 on Day 1, received the first cycle of treatment with initial improvement of symptoms, followed by disease progression and discontinuation from the study.

Patient 3, who received lintuzumab-Ac225 on Day 5, achieved a CRi (recovery in neutrophils but not platelets) after cycle 1 with normocellular BM and no increased blasts by flow. Next generation sequencing at the end of Cycle 1 was negative for known IDH2, RUNX1, SRSF2, and STAG2, but persistent for ASXL, TP53, and BRAF mutations.

These results support the superiority of otherwise comparable regimens in which lintuzumab-Ac225 is administered several days after daily venetoclax administration has begun.

The words “comprising” and forms of the word “comprising” as well as the word “including” and forms of the word “including,” as used in this description and in the claims, do not limit the inclusion of elements beyond what is referred to. Additionally, although throughout the present disclosure various embodiments or elements thereof are described in terms of “including” or “comprising,” corresponding embodiments or elements thereof described in terms of “consisting essentially of” or “consisting of” are similarly disclosed. For example, while certain embodiments of the invention have been described in terms of a method “including” or “comprising” administering a radiolabeled antibody, corresponding methods instead reciting “consisting essentially of” or “consisting of” administering the radiolabeled antibody are also within the scope of said aspects and disclosed by this disclosure. In this context, “consisting essentially of” provides a scope permitting the inclusion of further elements that do not materially affect the basic and novel or functional characteristic(s) of what is referred to, while the term “consisting of” excludes any element beyond what is referred to.

Any and all publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s). Moreover, features described in connection with one embodiment of the invention may be used in conjunction with other embodiments, even if not explicitly exemplified in combination within.

### REFERENCES

1. FDA News Release, FDA approves new drug for chronic lymphocytic leukemia in patients with a specific chromosomal abnormality, Apr. 11, 2016.
2. Venclexta™ Product Monograph Including Patient Medication Information (2016).
3. G. Kroemer, et al., Classification of Cell Death: Recommendations of the Nomenclature Committee on Cell Death 2009. Cell Death and Differentiation (2009) 16, 3-11.
4. S. Fulda, Tumor resistance to apoptosis, Int. J. Cancer: 124, 511-515 (2009).
5. E. Shiozaki, et al., Mechanism of XIAP-Mediated Inhibition of Caspase-9. Molecular Cell, Vol. 11, 519-527, February 2003.
6. I. Tamm, et al., Expression and prognostic significance of IAP-family genes in human cancers and myeloid leukemias. Clin Cancer Res. 2000; 6(5):1796-1803.
7. D. Potter and A. Letai, To Prime, or Not to Prime: That Is the Question, Cold Spring Harbor Symposia on Quantitative Biology, Volume LXXXI, Nov. 3, 2016.
8. Venetoclax Advisory Committee Briefing Document, Jun. 28, 2016.
9. J. Kiang, et al., Radiation Combined Injury: DNA Damage, Apoptosis, and Autophagy. Adaptive Medicine 2(1): 1-10, 2010.
10. C. Friesen, et al., Breaking Chemoresistance and Radioresistance with [²¹³Bi]anti-CD45 Antibodies in Leukemia Cells, Cancer Res 2007, 67(5): 1950-8.
11. S. Sofou, Radionuclide carriers for targeting of cancer, International Journal of Nanomedicine 2008:3(2), 181-199.
12. S. O'Steen, et al., Venetoclax Synergizes with Radiation Therapy for Treatment of B-Cell Lymphomas, ASH Annual Meeting 2016, Abstract 467.
13. R. Wilder, et al., International Prognostic Index-Based Outcomes for Diffuse Large B-Cell Lymphomas. Cancer Jun. 15, 2002/Volume 94/Number 12.
14. V. Bourke, et al., Correlation of Radiation Response with Tumor Oxygenation in the Dunning Prostate R3327-AT1 Tumor. Int. J. Radiat. Oncol. Biol. Phys. 2007 Mar. 15; 67(4): 1179-1186.
15. P. Vaupel, Tumor microenvironmental physiology and its implications for radiation oncology. Seminars in Radiation Oncology, Vol. 14, Issue 3, July 2004.
16. L. Harrison, et al., Hypoxia and Anemia: Factors in Decreased Sensitivity to Radiation Therapy and Chemotherapy? The Oncologist 2004, 9 (Suppl. 5), 31-40.
17. M. Hockel, et al., Tumor Hypoxia: Definitions and Curr. Clinical, Biologic, and Molecular Aspects, Journal of the National Cancer Institute, Vol. 93, No. 4, Feb. 21, 2001.
18. Zevalin® U.S.A. Package Insert (2001).
19. M. Roscher, et al., Targeted alpha-therapy using [Bi-213]anti-CD20 as novel treatment option for radio- and chemoresistant non-Hodgkin lymphoma cells, Oncotarget, February 2013, Vol. 4, No 2.
20. A. Konishi, et al., Involvement of Histone H1.2 in Apoptosis Induced by DNA Double-Strand Breaks. Cell, Vol. 114, 673-688, Sep. 19, 2003.
21. J. Stap, et al., Induction of linear tracks of DNA double-strand breaks by alpha-particle irradiation of cells. Nat. Methods, 2008 Mar. 5(3):261-6. doi: 10.1038/nmeth.f.206.
22. Pogozelski, et al., Quantitative assessment of the contribution of clustered damage to DNA double-strand breaks induced by 60Co gamma rays and fission neutrons. Radiat. Res. 1999 April, 151(4):442-8.
23. F. Graf, et al., (2014), DNA Double Strand Breaks as Predictor of Efficacy of the Alpha-Particle Emitter Ac-225 and the Electron Emitter Lu-177 for Somatostatin Receptor Targeted Radiotherapy. PLOS ONE 9(2): e88239. doi:10.1371/journal.pone.0088239.
24. P. A. Jeggo and M. Lobrich, DNA double-strand breaks: their cellular and clinical impact? Oncogene (2007) 26, 7717-7719.
25. D. Cicic, International Publication No. WO 2018/200841.

1. A method for treating a human subject afflicted with acute myeloid leukemia comprising administering to the subject a regimen of venetoclax and lintuzumab-Ac225, wherein (a) the regimen comprises a plurality of cycles, each cycle lasting from 28 to 60 days, (b) the regimen comprises (i) orally administering venetoclax on days 1 and 2 of the first cycle at a ramp-up dosage, and thereafter orally administering 400 mg of venetoclax daily on days 3-21 of the first cycle and days 1-21 of each subsequent cycle, and (ii) intravenously administering lintuzumab-Ac225 on day 4, 5, or 6 of each cycle at a dose of from 0.1 μCi/kg to 2.0 μCi/kg; and (c) the subject has a peripheral blast burden at or below 1,000 blast cells/μl.

2. The method of claim 1, wherein the regimen comprises four cycles.

3. The method of claim 1, wherein the regimen comprises eight cycles.

4. The method of claim 1, wherein each cycle lasts 28 days.

5. The method of claim 1, wherein the ramp-up dosages of venetoclax on days 1 and 2 of the first cycle are 100 mg and 200 mg, respectively.

6. The method of claim 1, wherein the lintuzumab-Ac225 dose is 0.25 μCi/kg, 0.5 μCi/kg, 1.0 μCi/kg, or 1.5 μCi/kg.

7. A method for treating a human subject afflicted with acute myeloid leukemia comprising administering to the subject a regimen of venetoclax and lintuzumab-Ac225, wherein (a) the regimen comprises four cycles, each cycle lasting 28 days, (b) the regimen comprises (i) orally administering venetoclax at a dose of 100 mg and 200 mg, respectively, on days 1 and 2 of the first cycle, and thereafter orally administering 400 mg of venetoclax daily on days 3-21 of the first cycle and days 1-21 of each subsequent cycle, and (ii) intravenously administering lintuzumab-Ac225 on day 5 of each cycle at a dose of 0.25 μCi/kg, 0.5 μCi/kg, 1.0 μCi/kg, or 1.5 μCi/kg; and (c) the subject has a peripheral blast burden at or below 1,000 blast cells/μl.

8. A method for treating a human subject afflicted with acute myeloid leukemia comprising administering to the subject a regimen of venetoclax and lintuzumab-Ac225, wherein (a) the regimen comprises a plurality of cycles, each cycle lasting from 28 to 60 days, (b) the regimen comprises (i) orally administering venetoclax on days 1 and 2 of the first cycle at a ramp-up dosage, and thereafter orally administering 400 mg of venetoclax daily on days 3-21 of the first cycle and days 1-21 of each subsequent cycle, and (ii) intravenously administering lintuzumab-Ac225 on day 4, 5, or 6 of each cycle at a dose of from 0.05 μCi/kg to 1.0 μCi/kg, and on day 18, 19, or 20 of each cycle at a dose of from 0.05 μCi/kg to 1.0 μCi/kg; and (c) the subject has a peripheral blast burden at or below 1,000 blast cells/μl.

9. The method of claim 8, wherein the regimen comprises four cycles.

10. The method of claim 8, wherein the regimen comprises eight cycles.

11. The method of claim 1, wherein each cycle lasts 28 days.

12. The method of claim 1, wherein the ramp-up dosages of venetoclax on days 1 and 2 of the first cycle are 100 mg and 200 mg, respectively.

13. The method of claim 1, wherein the lintuzumab-Ac225 dose is 0.125 μCi/kg, 0.25 μCi/kg, 0.5 μCi/kg, or 0.75 μCi/kg.

14. A method for treating a human subject afflicted with acute myeloid leukemia comprising administering to the subject a regimen of venetoclax and lintuzumab-Ac225, wherein (a) the regimen comprises four cycles, each cycle lasting 28 days, (b) the regimen comprises (i) orally administering venetoclax at a dose of 100 mg and 200 mg, respectively, on days 1 and 2 of the first cycle, and thereafter orally administering 400 mg of venetoclax daily on days 3-21 of the first cycle and days 1-21 of each subsequent cycle, and (ii) intravenously administering lintuzumab-Ac225 on day 5 of each cycle at a dose of 0.125 μCi/kg, 0.25 μCi/kg, 0.5 μCi/kg, or 0.75 μCi/kg, and on day 19 of each cycle at a dose of 0.125 μCi/kg, 0.25 μCi/kg, 0.5 μCi/kg, or 0.75 μCi/kg; and (c) the subject has a peripheral blast burden at or below 1,000 blast cells/μl.


SEQUENCE LISTING (4 sequences)

SEQ ID NO: 1 - DNA, 1952 bp, Artificial Sequence
  Description of Artificial Sequence Synthetic polynucleotide
tctagaccaccatggagaaagacacactcctgctatgggtcctacttctctgggttccaggttccacaggtgacattcagatgacccagtctccgagctctctgtccgcatcagtaggagacagggtcaccatcacatgcagagccagcgaaagtgtcgacaattatggcattagctttatgaactggttccaacagaaacccgggaaggctcctaagcttctgatttacgctgcatccaaccaaggctccggggtaccctctcgcttctcaggcagtggatctgggacagacttcactctcaccatttcatctctgcagcctgatgacttcgcaacctattactgtcagcaaagtaaggaggttccgtggacgttcggtcaagggaccaaggtggagatcaaacgtaagtagaatccaaagtctagaaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgctctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtggtttctctctttcctcatttaataattattatctgttgttttaccaactactcaatttctcttataagggactaaatatgtagtcatcctaaggcgcataaccatttataaaaatcatccttcattctattttaccctatcatcctctgcaagacagtcctccctcaaacccacaagccttctgtcctcacagtcccctgggccatggtaggagagacttgcttccttgttttcccctcctcagcaagccctcatagtcctttttaagggtgacaggtcttacagtcatatatcctttgattcaattccctgagaatcaaccaaagcaaatttttcaaaagaagaaacctgctataaagagaatcattcattgcaacatgatataaaataacaacacaataaaagcaattaaataaacaaacaatagggaaatgtttaagttcatcatggtacttagacttaatggaatgtcatgccttatttacatttttaaacaggtactgagggactcctgtctgccaagggccgtattgagtactttccacaacctaatttaatccacactatactgtgagattaaaaacattcattaaaatgttgcaaaggttctataaagctgagagacaaatatattctataactcagcaatcccacttctaggatcc

SEQ ID NO: 2 - protein, 238 aa, Artificial Sequence
  Description of Artificial Sequence Synthetic polypeptide
MEKDTLLLWVLLLWVPGSTGDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 3246 bp, Artificial Sequence
  Description of Artificial Sequence Synthetic polynucleotide
tctagaccaccatgggatggagctggatctttctcttcctcctgtcaggaactgctggcgtccactctcaggttcagctggtgcagtctggagctgaggtgaagaagcctgggagctcagtgaaggtttcctgcaaagcttctggctacaccttcactgactacaacatgcactgggtgaggcaggctcctggccaaggcctggaatggattggatatatttatccttacaatggtggtaccggctacaaccagaagttcaagagcaaggccacaattacagcagacgagagtactaacacagcctacatggaactctccagcctgaggtctgaggacactgcagtctattactgcgcaagagggcgccccgctatggactactggggccaagggactctggtcactgtctcttcaggtaagaatggcctctagaccaccatgggatggagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagcccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagggaggcagagcgggtcccactgtccccacactggcccaggctgtgcaggtgtgcctgggccgcctagggtggggctcagccaggggctgccctcggcagggtgggggatttgccagcgtggccctccctccagcagcacctgccctgggctgggccacgggaagccctaggagcccctggggacagacacacagcccctgcctctgtaggagactgtcctgttctgtgagcgccctgtcctccgacctccatgcccactcgggggcatgcctagtccatgtgcgtagggacaggccctccctcacccatctacccccacggcactaacccctggctgccctgcccagcctcgcacccgcatggggacacaaccgactccggggacatgcactctcgggccctgtggagggactggtgcagatgcccacacacacactcagcccagacccgttcaacaaaccccgcactgaggttggccggccacacggccaccacacacacacgtgcacgcctcacacacggagcctcacccgggcgaactgcacagcacccagaccagagcaaggtcctcgcacacgtgaacactcctcggacacaggcccccacgagccccacgcggcacctcaaggcccacgagcctctcggcagcttctccacatgctgacctgctcagacaaacccagccctcctctcacaagggtgcccctgcagccgccacacacacacaggggatcacacaccacgtcacgtccctggccctggcccacttcccagtgccgcccttccctgcaggatcc

SEQ ID NO: 4 - protein, 465 aa, Artificial Sequence
  Description of Artificial Sequence Synthetic polypeptide
MGWSWIFLFLLSGTAGVHSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK